(19) United States
(12) Patent Application Publication
(10) Pub. No.: US 2011/0293537 A1
(43) Pub. Date: Dec. 1, 2011

(54) FORMULATIONS WITH ANTI-NEOPLASTIC ACTIVITY

(76) Inventors: Giovanni Nicolao Berta, Valperga Canavese (TO) (IT); Paolina Salamone, Pasta di Rivalta (TO) (IT); Andrea Sprio, Torino (IT); Maria Eugenia Carlotti, Torino (IT); Simona Sapino, Brandizzo (TO) (IT); Roberta Cavalli, Alessandria (IT); Silvia Marino, Trofarello (TO) (IT); Alessandro Barberis, Valgrana (CN) (IT); Barbara Mognetti, Torino (IT); Francesco Di Carlo, Torino (IT)

(21) Appl. No.: 13/130,912

(22) PCT Filed: Nov. 24, 2008
(86) PCT No.: PCT/IB08/03271

§ 371 Date: Aug. 8, 2011

(51) Int. Cl. A61K 31/724; A61K 9/68; A61P 17/12; A61P 17/00; A61P 17/06; A61K 31/7048; A61P 35/00

(52) U.S. Cl. 424/48; 514/58; 514/27

(57) ABSTRACT

Pharmaceutical formulation comprising at least one aryl hydrocarbon receptor ligand and at least one cyclodextrin for the prevention and/or treatment of carcinomas, preferably squamous cell carcinomas, more preferably oral squamous cell carcinomas.

### FIELD OF THE INVENTION

The present disclosure concerns a new pharmaceutical formulation with anti-neoplastic activity.

### BACKGROUND OF THE INVENTION

Despite of the progress obtained in anticancer therapies, 7.6 of 58 millions worldwide deaths are ascribed to neoplasms in 2005. OMS estimates these data will increase to 9 millions in 2015 and to 11.4 million in 2030.

These data are heavily influenced since some cancer hystotypes are only partially or not at all responsive to the therapeutic strategies currently available and characterized by a poor prognosis.

One of this is represented by Squamous Cell Carcinomas (SCC): approximately 1 million cases of non-melanoma cancers of the Stratified Squamous Epithelia (SSE) are identified each year. SSE are found in cervix, skin and oral cavity.

Among SCC, particularly worrying is the oral situation: the worldwide incidence of Oral Squamous Cell Carcinoma (OSCC) is globally stable in the last decades because of the lack of effective therapies. Furthermore, OSCC is a complex disease because its location and patterns of spread provide unique challenges for oncologist and the surgery is still the therapy of choice. Typically, OSCC development is a slow and cumulative process, which occurs after exposure of the entire epithelial surface to the repeated insult of carcinogens. This concept was introduced by Slaughter in 1953, and was based on the fact that the epithelial surface of the aero-digestive tract is likely to be exposed to many of the common carcinogens such as tobacco and alcohol and thus has an increased risk of OSCC development. Five years after treatment of first OSCC, up to 20% of patients have developed a second tumor toward which the therapeutic strategies are very limited or exhausted reporting a survival rate of 20% at 1 year. Despite of improvements in plastic and reconstructive techniques, surgery often leaves patients with chronic pain, loss of function (particularly with speech and swallowing) and irreparable, socially disfiguring impairment. The functional, cosmetic and psychological repercussions suffered by oral cancer patients often result in social isolation, significantly burdening patients, their families and society. Generally, their nutritional status is also poor and depression occurs frequently in these patients.

The OSCC is usually preceded by the presence of clinically identifiable pre-malignant changes of the oral mucosa (OPL: oral premalignant lesion). This kind of lesions is common, with a prevalence of about 1% in the population and a high potential of neoplastic transformation (20%-40%). Oral leukoplakia is the most common OPL of OSCC and up to 20% of the patients with leukoplakia develop invasive carcinoma. Although the diagnosis should be facilitated due to the accessibility of oral cavity, currently they lack any effective therapeutic approach to regress or stop the neoplastic transformation. This significantly affects the psychological aspect of patients, because the morbid process not immediately represents a risk for their survival, but it should became in the next future.

The alarming epidemiological data, the poor prognosis, the unsatisfactory quality of life and the lack of effective therapies for patients with OPL or OSCC, render the development of novel approaches to treat these diseases compelling.

During the last years, a series of preclinical and clinical studies, based on chemoprevention approaches, has been performed but the clinical results are unsatisfactory. Chemoprevention might be a tool to fulfil this strategy and to offer a promising treatment for these kinds of disease, since the multi-step carcinogenesis process strongly supports the rationale for a preventive strategy that will inhibit, delay, or reverse carcinogenesis before it becomes an invasive disease. Generally, systemic chemopreventive agents, while effective, have a limited clinical utility due to significant side effects.

The localization of oral cavity and its easy accessibility offer a unique opportunity for the development of local/topical chemopreventive approaches. In the last decade, many plant-based agents are recognized to exert their anticarcinogenic effects, with promising results. Unfortunately, most of them were not able to confirm their antineoplastic activity in vivo for unfavorable phamacokinetic properties and/or presence of unacceptable side effects.

Between these molecules, one of the most studied for its anticancer activity is resveratrol (3,4′,5-trihydroxy-trans-stilbene), an aryl hydrocarbon receptor (AhR) antagonist/ligand produced by plants in response to a parasitic attack or under stress conditions. It is found in appreciable amount in a variety of edible fruits including nuts, berries, grape skin and consequently in wine.

Resveratrol (RV), like other aryl hydrocarbon receptor antagonists, plays in human health many activities, such as antioxidant, lipoprotein metabolism modulator, platelet aggregation inhibitor, antifungal, antiviral, phytoestrogenic and cardioprotector.

In particular, RV has shown strong antineoplastic activities in many neoplastic cell lines (principally obtained from colon, skin, breast, lung, prostate, liver and pancreas) as confirmed by many papers published in the last years. This effect is due to inhibition of cell proliferation, induction of apoptosis and arrest of cell-cycle progression. In its activity RV is responsible of alterations in more than one hundred pathways, such as transcriptional inhibition of some cytochrome P450 isoforms and NF-κB factor, expression and activity regulation of cyclooxygenase enzymes and activation of SIRT1. Moreover, RV has been shown to induce Fas/Fas ligand-mediated apoptosis, p53, and cyclin kinase inhibitor WAF1/CIP1/p21, which are able to inhibit cyclin D1 and cyclin E, and their regulatory subunits cdk2 and cdk6 with a decrease of hyperphosphorilation of pRb, E2F transcription factors release and induction of arrest of cell cycle in G₀-G₁ phase.

Although RV has attracted considerable attention for clinical trials because serious adverse events are not observed after its systemic administration, unfavorable results were obtained due to its unsatisfactory pharmacokinetic properties. Despite of its high oral absorption (at least 70%), RV has a very short initial half-life (8-14 min), because it is metabolized very quickly and extensively in the body resulting in only trace amounts (less than 2%) of unchanged RV. The vast majority (>95%) of RV is in fact conjugated as glucuronides and sulphates by second order metabolism reactions. In particular, RV oral bioavailability in human is observed to be very poor, leading to an irrelevant in vivo effect compared to its remarkable in vitro efficacy. Ingestion by humans of 25 mg (approximately 0.1 mmol) RV yields systemic levels of 7.5 to 40 nmol/L, while a concentration at least 5 μmol/L RV is required to obtain appropriate chemoprevention.

### SUMMARY OF THE INVENTION

The need is therefore felt for the identification of new pharmaceutical formulations effective against carcinomas for the prevention and/or treatment of carcinomas, preferably skin and/or mucosa carcinomas.

The object of this disclosure is providing such formulations.

According to the invention, the above object is achieved thanks to the subject matter recalled specifically in the ensuing claims, which are understood as forming an integral part of this disclosure.

An embodiment of the present disclosure provides a new pharmaceutical formulation comprising at least one aryl hydrocarbon receptor ligand and at least one cyclodextrin for the prevention and/or treatment of carcinomas, preferably squamous cell carcinomas, more preferably oral squamous cell carcinomas.

Topical administration of an aryl hydrocarbon receptor ligand through skin and oral mucosa is, in fact, a way for an easier accessibility of the anatomical sites involved (skin, oral cavity, esophagus and stomach) and to obtain locally sufficient therapeutic concentration avoiding the systemic degradation (due to the low metabolism in epithelial tissue) with an improvement of the aryl hydrocarbon receptor ligand half-life and an improvement of their anti-neoplastic activity.

### BRIEF DESCRIPTION OF THE DRAWINGS

The invention will now be described, by way of example only, with reference to the enclosed figures of drawing, wherein:

FIG. 1. Schedule of experiment. Three-time a week (on Monday, Wednesday and Friday—gray arrows) carcinogenesis was induced through 9,10-dimethyl-1,2-benz[a]anthracene (DMBA) painting on right buccal pouches for all animals of all groups: this was the only treatment for “super-control” group (D). Otherwise, on Tuesday and Thursday treated RV-Fs (resveratrol formulations) groups (resveratrol dissolved in EtOH (ER), resveratrol complexed with ciclodextrin in cream (CR) and resveratrol complexed with ciclodextrin in mouthwash (MR)) and correspondent control groups (EtOH (E), cream (C) and mouthwash (M)) were treated with RV-Fs or respective vehicle alone (white arrows). Since 4^{th} week hamsters right pouches was weekly examined (asterisks) and any lesions present evaluated. Animals were sacrificed at 14^{th} week (black arrow).

FIG. 2. in vitro vehicles toxicity. Graphic representation of MTT assay on HCPC1 cells incubated for 72 h with increasing dilutions of ethanol (panel A), cream (panel B) or mouthwash (panel C). Significant antiproliferative action of ethanol was achieved since 1:100 dilution (170 mM), whereas analogue effect was not observed until 1:10 dilution (hydroxypropyl-β-cyclodextrin (HEβCD) 16.6 mM) for cream and mouthwash.

*=P<0.05; **=P<0.01; ***=P<0.001 vs control.

FIG. 3. in vitro RV-Fs antiproliferative action. Graphic representation of MTT assay on HCPC1 cells incubated for 24, 48 and 72 h with increasing concentration of RV-Fs. RV ethanol solution EC₅₀ was achieved only after 72 h at 45 μM concentration (panel A). Cream and mouthwash complexing RV had reached EC₅₀ already at 48 h with a concentration of 70 and 45 μM respectively, while at 72 h EC₅₀ was 20 μM both (panel B and C).

*=P<0.05; **=P<0.01; ***=P<0.001 vs control.

FIG. 4. Hamster weight over experimentation. No significant differences in body weight were observed between all groups.

FIG. 5. Lesions incidence. Graphic representations of the number of animals displayed lesions (including OPL and exophytic lesions (ExL)) during 4^{th}-14^{th} weeks of treatment. RV treated groups displayed a delay in their appearance: if statistically compared with respective control groups (D and M), MR animals developed lesions later until 12^{th} week (P<0.05) shown the best efficacy.

FIG. 6. Lesions prevalence. Since 9^{th} week, control groups animals generally developed a statistically greater number of lesions compared to those found in hamsters treated with RV-Fs. At the end of experiments, each animals of control groups displayed about 9 lesions while RV-treated ones developed only 3 (ER and CR P<0.01; MR P<0.001). Analogously to incidence, MR group also proved more effectiveness than ER and CR (P<0.05).

FIG. 7. Oral premalignant lesions incidence. From the 5^{th} week a growing number of animals developed OPL. In RV-treated groups, the number of hamsters affected was drastically reduced if compared with controls (7-11^{th} week three-fold lesser, D vs ER, CR and MR, E vs ER, C vs CR and M vs MR p<0.05).

FIG. 8. Oral premalignant lesions prevalence. OPL multiplicity was found significant from 7^{th} to 11^{th} week between RV-Fs groups and respective control ones and vs DMBA only treated animals (D vs ER P<0.05; D vs CR and MR P<0.01; E vs ER P<0.01; C vs CR and M vs MR P<0.05).

FIG. 9. Exophytic lesions incidence. ExL incidence was the less influenced parameter by RV treatment; in few cases there were significant differences between groups: D vs MR at 10^{th} week (P<0.01); C vs CR at 8^{th} and 11^{th} week (P<0.05); M vs MR at 10^{th} and 12^{th} week (P<0.05).

FIG. 10. Exophytic lesions prevalence. ExL multiplicity was strongly influenced by RV treatment already at 10^{th} week: MR group showed a significant reduction respect D and M groups (P<0.01). In ER and CR groups, differences respect control were significant from 12^{th} week (P<0.05). RV action reduced more than three fold the ExL number if compared with ones in control groups.

FIG. 11. Exophytic lesions dimension. Average lesions diameter was largely influenced by RV treatment: MR group ExL were 10-fold smaller if compared with D and M (P<0.001) and about half size respect ER and CR ones (P<0.05). Moreover ExL size of ER and CR was 50% of respective controls (P<0.05).

FIG. 12. Pathological score (PS). PS expressed the overall severity of lesions contextually diagnosed in a single buccal pouche (for the criteria see text). RV-treated groups showed a significant PS decrease (after the 10^{th} week, MR vs D and M P<0.01; MR vs ER and CR P<0.05). Two weeks later, also ER and CR demonstrated a better situation respect control groups (P<0.05).

FIG. 13. HE histological analysis. Comparison of typical histological alterations of buccal pouches in RV-treated animals (panel A) and control ones (panel B). Frequently, RV-Fs groups developed only papilloma like lesions, while generally frankly invasive carcinoma were found in control groups. (100× magnification).

### DETAILED DESCRIPTION OF EXEMPLARY EMBODIMENTS

In the following description, numerous specific details are given to provide a through understanding of embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The headings provided herein are for convenience only and do not interpret the scope or meaning of the embodiments.

The present disclosure concerns a new pharmaceutical formulation comprising at least one molecule able to bind to aryl hydrocarbon receptor preventing aryl hydrocarbon binding (preferably aryl hydrocarbon receptor antagonist, more preferably resveratrol) in association with at least one cyclodextrin (CD) (preferably water-soluble cyclodextrin, more preferably hydroxypropyl-β-cyclodextrin).

The compounds able to bind to aryl hydrocarbon receptor of interest in the present disclosure are stilbene derivates and flavone derivates of formula I and formula II, respectively:

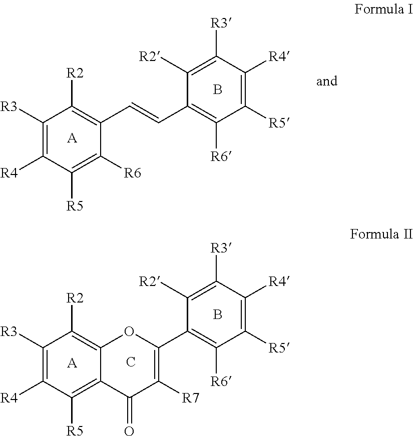

wherein
R2, R3, R4, R5, R6, R7 and R2′ R3′, R4′, R5′, R6′ are identical or different (including all symmetrical derivatives) and represent H, OH, R (where R represents substituted or unsubstituted, saturated or unsaturated, linear or branched aliphatic groups containing one to thirty carbon atoms), Ac (where Ac represents substituted or unsubstituted, saturated or unsaturated, cyclic compounds, including alicyclic and heterocyclic, preferably containing three to eight atoms), Ar (where Ar represents substituted or unsubstituted, aromatic or heteroaromatic groups preferably containing five or six atoms), Cr (where Cr represents substituted or unsubstituted fused Ac and/or Ar groups, including Spiro compounds and norbornane systems, preferably containing two to five fused rings), OR, X (where X represents an halogen atom), CX₃, CHX₂, CH₂X, glucoside, galactoside, mannoside derivates, sulfate and glucuronide conjugates.

All optical and geometrical isomeric derivatives of stilbene and flavone compounds are included. In addition plant extracts containing one of these molecules must be included.

Among the compounds encompassed by the general formulas I and II, one can cite apigenin (4′,5,7-trihydroxyflavone), luteolin (3′,4′,5,7-tetrahydroxyflavone), tangeritin (4′,5,6,7,8-pentamethoxyflavone), diosmin (5-Hydroxy-2-(3-hydroxy-4-methoxyphenyl)-7-[(2S,3R,4S,5S,6R)-3,4,5-trihydroxy-6-[[(2R,3R,4R,5R,6S)-3,4,5-trihydroxy-6-methyloxan-2-yl]oxymethyl]oxan-2-yl]oxychromen-4-one), flavoxate (2-(1-piperidyl)ethyl 3-methyl-4-oxo-2-phenyl-chromene-8-carboxylate), piceatannol (3,4,3′,5′-tetrahydroxystilbene), oxyresveratrol (2,3′,4,5′-tetrahydroxystilbene), 4,4′-dihydroxystilbene.

Although some of these molecules may act as agonist of AhR, often their activity does not involve all AhR signal transduction cascades, antagonizing, hence, pollutants and condensed polycyclic hydrocarbons activity. Since AhR molecular pathways activated through receptor binding are not still clear, a paramount of aryl hydrocarbon receptor ligands are useful for the present purpose.

The expression “aryl hydrocarbon receptor ligand” means every molecule (preferably all stilbene and flavone derivate structures) with partial or complete agonist or antagonist activity that binds an AhR, preventing its occupation by condensed polycyclic hydrocarbons.

Since the number of cyclodextrins known is impressive and very large, because little differences between all congeners are numerous, the present formulations can include α-, β- and γ-natural CD, and all their neutral, anionic or cationic semi-synthetic and synthetic derivates with different degree of substitution (in particular full or partial substitution of hydroxyl groups by various functions), both hydrophilic and lipophilic ones and their salts. Cyclodextrins encompassed by the present disclosure may be in monomeric or polymeric forms, crystalline or amorphous or mixtures of them, and also CD linked with any type of polymers, supramolecular structures including molecular capsules, spheres and in general all CD self-associated complexes to form micro- and nano-scale aggregates, including novel surface active CD derivatives and CD-based nanosponges or simply “nanosponges”. All optical and geometrical isomeric derivates are encompassed by the instant disclosure. Some examples of the most used CD are: the hydroxyalkylated CD such as 2-hydroxypropyl derivatives of both β- and γ-cyclodextrin (HPβCD and HPγCD respectively), the sulphobutylether derivative of β-cyclodextrin (SBβCD) and its sodium salt, the branched glucosyl- and maltosyl-β-cyclodextrins (particularly G2βCD or 6-O-Maltosyl-β-cyclodextrin), the randomly methylated β-cyclodextrin (RMβCD), the cationic CD (for example the simplest mono-6 or 2-amino-β-CD hydrochlorides), but also mono-6-amino-6-deoxy-α- or γ-cyclodextrin hydrochlorides, disubstituted β-CD derivatives, hepta-2,3-hepta-2,6-di- and hepta-2,3,6-tri-substituted β-CD derivates, hepta-6-S-6-deoxy-β-CD derivatives, mono-6-tosyl-β-CD, mono-6-alkylimidazolium-β-CDs, 6-O-carboxymethyl-β-CD.

Among the stilbene derivates and flavone derivates of formula I and formula II object of the instant application, particular attention, without any limiting purpose on the present application, has been devoted to resveratrol, a stilbene derivative of formula I (3,4′,5-trihydroxy-trans-stilbene). Reseveratrol is one of the possible embodiments of the instant application.

Serious adverse events have not been observed after resveratrol systemic administration and topical application is an ideal way to minimize adverse reactions. The present data suggest a good tolerance of topical RV application on oral mucosa.

With respect to CD, all toxicity studies have demonstrated that orally administered HPβCD is practically non-toxic, due to lack of absorption from the gastrointestinal tract and is well tolerated in humans. The main adverse event has been observed only with high doses (16-24 g HPβCD/day) for almost 14 days with increased incidents of soft stools and diarrhea only. Actually worldwide 5 different HPβCD-containing pharmaceutical products are marketed: Prepulsid® (cisapride, Janssen), Dexocort® (hydrocortisone, Actavis), Indocid (indomethacin, Chauvin), Sporanox® (itraconazole, Janssen) and MitoExtra™ (mitomycin, Novartis) differently formulated (oral, i.v., eye drop solutions and suppository).

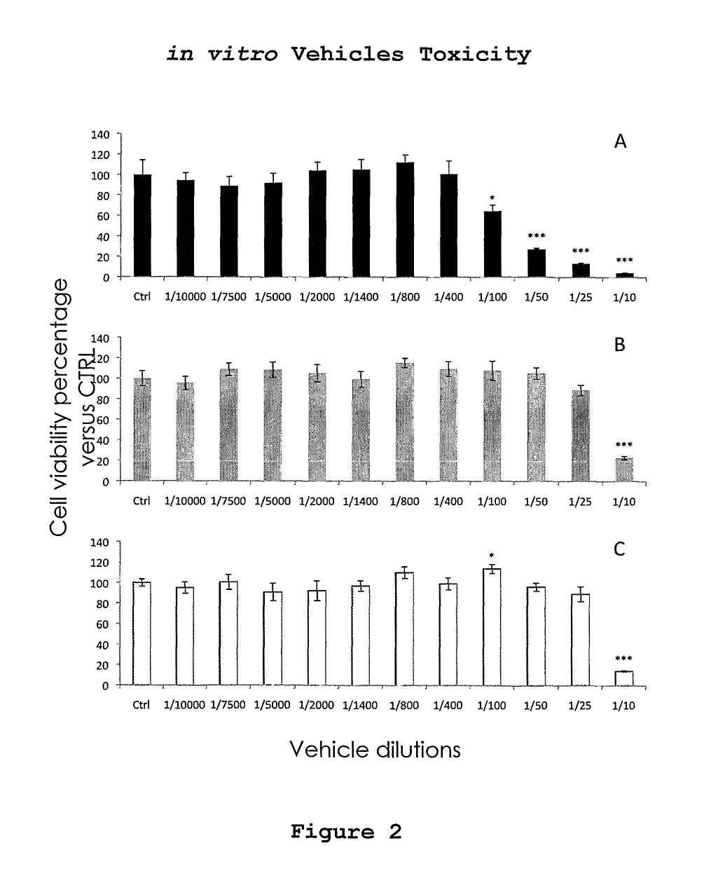

In the present disclosure HPβCD and their formulations complexing RV lack toxicity: HPβCD in vitro experiments showed inhibition of cell growth only at extremely high concentrations (see FIG. 2) probably due to physical phenomena (viscosity/osmotic pressure changes). The concentrations adopted in the present in vivo experiments (and generally clinically used) were ranged about a thousand fold less and they did not display any toxic signs or adverse effects on the HPβCD chronic treated hamsters.

Regarding RV antineoplastic action, RV is able to operate as chemopreventive molecule in all three stages of cancer development (initiation, promotion and progression) including OSCC one. In the present disclosure, RV has shown the ability to inhibit the in vitro HCPCI proliferation, OSCC growth and, interestingly, preneoplastic lesions in hamster model.

Hamster buccal pouche is one of the best known animal systems to investigate OPS, and OSCC development and to intervene by chemopreventive agents.

DMBA, an aryl hydrocarbon, is one of the reference compounds most often used in mutagenesis and experimental multistep carcinogenesis. Aromatic polycyclic induction fits OSCC development in human, whose aetiology has been linked to environmental factors and epidemiological studies strongly suggest a role for chemical carcinogens. Tobacco and alcohol are also possible etiologic factors that may synergistically induce approximately 75% of SCC in the oral mucosa and larynx.

The present data have confirmed the strong anti-proliferative and chemo-preventive action of RV since the DMBA induction of carcinogenesis is very powerful, although it is able to interfere with more than one hundred cellular pathways, being RV a potent competitive aryl hydrocarbon receptor antagonist. A lot of condensate polycyclic aromatic hydrocarbons (including benzo[a]pyrene and benz[a]anthracene derivates), present in high concentration in cigarette smoke, bind with AhR and enhance gene transcription of xenobiotic-metabolizing enzymes such as cytochrome P450 CYP1A1 and 1B1 isoforms, glutathione-S-transferases (GST) and NAD(P)H:quinone reductase (NQO1). P450 isoforms activate benzo[a]pyrene and benz[a]anthracene derivates promoting their DNA adducts formation leading to cancer initiation.

RV action prevents the formation of active forms of polycyclic aromatic hydrocarbons, mainly inhibiting the activity of cytochrome P450 1B1.

Another advantage of the disclosed therapeutical approach lies in topical application of the formulations according to the present disclosure because bioavailability is improved due to circumvention of systemic degradation and a low metabolism in mucosa tissue.

Moreover, non-ionic RV form shows high lipophilicity and is able to permeate mucosa keratinized strata easily. RV dissociation level is dependent upon its pKa (9.3, 10, 10.6 for the 4′-3- and 5-OH groups respectively) and upon the pH of the aqueous environment: saliva pH ranges between 5-8 and for these values RV is almost all present in its neutral form and, consequently, it is capable to cross biological membranes.

Regarding the efficacy of HPβCD complexing RV, they have shown an improved anti-neoplastic action with respect to RV alone, both in vitro and in vivo experiments: surprisingly, mouthwash (MR) formulation demonstrated the best effect for all considered parameters, while cream (CR) formulation has shown intermediate results with respect to RV simply dissolved in EtOH. About this aspect, probably physical properties of solution containing CDs are able to enhance local RV delivery with respect to cream.

Generally, drug oral topical applications can be easily and quickly removed by different mechanism such as wetting (by saliva, beverage), movement (e.g. tongue, muscles) or contact (e.g. food) with consequent efficacy reduction or action lack.

CD provide an ideal way to retain RV on oral mucosa or skin through enhanced adhesion for a significant period of time. Hydrophilic CD, such as HPβCD, are unable to permeate biological membranes (including the ones of skin or mucosa) and to enhance permeation of hydrophilic drugs, but they generally show paramount advantages when they complex hydrophobic ones, making them an ideal carrier to reach our purpose.

In summary HPβCD are: i) the most suitable for aromatic heterocyclic molecules hosting, such as RV; ii) able to enhance solubility of lipophilic drugs; iii) able to reduce drug permeation through lipophilic membranes by decreasing drug partition from the exterior into the membrane (a property useful to prevent mucosal absorption with an improvement of local action); iv) able to increase the bioavailability through chemical stabilization of complexed drugs and, finally, v) able to reduce the drug release from water/oil creams but enhances the release from oil/water creams (CR formulations).

It can be concluded that CD may be an ideal vehicle for RV delivery because provides high levels of drug permeation and mucosa safety.

Chemoprevention of early stage of the oral carcinogenesis process is important for management of patients because reduces OPL incidence and consequently OSCC development. Since HPβCD complexing RV showed an important chemopreventive action on preclinical experimentation, beneficial therapeutical approach are proposed to prevent OSCC progress in patients with OPL or already operated for a first OSCC (with a high risk of recurrent second or recidivant tumor). Furthermore, considering the lacking of adverse effect, RV may be proposed for people with high risk to develop OSCC due to smoking or/and drinking habit.

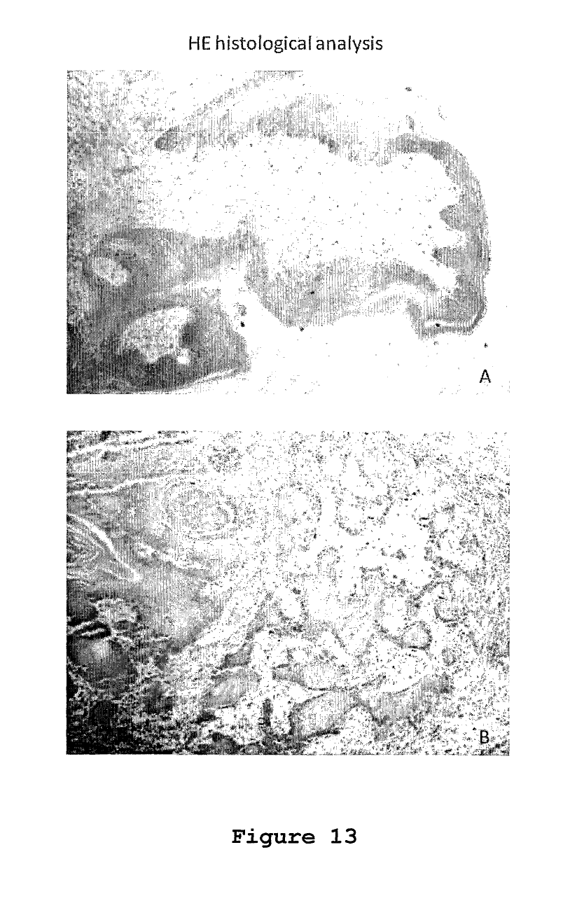

Histological analysis confirmed the antineoplastic activity of RV not only for considered lesions end-points (incidence, prevalence, dimensions, . . . ) but also for its capability to mitigate histological signs of malignancy in RV-Fs treated animals buccal pouches lesions (FIG. 13, Table 1).

Since an amount of applied formulation on oral cavity can reach esophageal and gastric districts, the applied formulations may provide for these anatomical sites tumors prevention. Due to the similarity of OPL and OSCC with cutaneous ones (cutaneous preneoplastic lesions, including actinic dyskeratosis, and cutaneous squamous cell carcinoma) there is the possibility to interfere also with them.

Finally, CD increase the bioavailability through chemical stabilization of drug molecules, hence RV may be administrated orally and the antineoplastic action is conceivable versus other systemic neoplastic sites.

Topical chemoprevention with RV/CD is a promising and realistic approach for clinicians and patients. In addition to fewer systemic side effects, the advantages of topical chemoprevention include easy application that does not require medical supervision and a relatively low cost compared with other interventions.

### Materials and Methods

### Formulations

Mouthwash (MR): 30% (250 mM) Kleptose®HPB (hydroxypropyl-β-cyclodextrin (HPβCD) from Roquette, Lestrem, France), 0.2% Kemipur® (imidazolidynyl urea from A.C.E.F., Fiorenzuola d'Arda, Italy), 0.3% Fenossiparaben (phenoxyethanol, methylparaben, ethylparaben, propylparaben and buthylparaben from Sinerga, Milan, Italy) were added, one by one to proper amount of physiological solution under manual stirring.

Cream (CR): 7% Finsolv®TN(C₁₂₋₁₅ alkylbenzoate from A.C.E.F.) was added to the above Mouthwash homogenizing using an Ultra Turrax® for 2 minutes at 37° C. Then 2% Sepigel® 305 (polyacrylamide, C₁₃₋₁₄ isoparaffin, laureth-7 from Seppic, Paris, France) was added and the mixture was homogenized for 2 minutes.

RV-formulations (MR and CR): to obtain RV/HPβCD complexes, under 24 h of magnetic stirring an opportune amount of RV (to reach concentration of about 100 mM) was dispersed in a physiological solution previously added with HPβCD. This solution was then used as above described to obtain the two formulations. RV loading was assessed in each formulation by HPLC analysis using a Shimadzu apparatus with a RPC18 column (80×4.6 mm, 5 μm). The mobile phase was a mixture of methanol/water/HCl (55/44.6/0.4) at a flow rate of 0.8 ml/min and the detection wavelength was 306 nm. The final concentration detected of both RV-Fs was 17 mg/ml (74.5 mM).

### Cell Lines

The HCPC-I cell line (kindly provided by Professor D.T. Wong, UCLA, Los Angeles, Calif., USA, and publicly available by University of Turin, Department of Clinic and Biologic Sciences, San Luigi Gonzaga Hospital, Orbassano, Italy), established from a chemically induced epidermoid carcinoma of the Syrian hamster [Odukoya et al, 1983], was cultured in 75 cm² TPP® flasks (TPP AG, Switzerland) in RPMI-1640 supplemented with 10% fetal calf serum (FCS), with 100 U/ml penicillin G, 40 μg/ml gentamicin sulphate and 2.5 μg/ml amphotericin B (all from Sigma-Aldrich, St Louis, Mo., USA) at 37° C. in a humidified 5% CO2 atmosphere. Cells were harvested every 3-4 days with 0.25% trypsin solution (from Sigma).

### Cytotoxicity Assay

Cytotoxicity was evaluated with MTT (3-(4,5-Dimethylthiazol-2-yl)-2,5-diphenyl-tetrazolium bromide) assay on 96-well TPP® plates (TPP AG). HCPC-I cells were dispensed at a density of 5×10²/100 μl for each well in RPMI-1640 supplemented with 10% FCS. After one day, culture medium was replaced and supplemented with serial concentrations of RV (Cabru, Arcore, Italy) dissolved in ethanol (EtOH, Sigma) or complexed with HPβCD, either in mouthwash (MR) or in cream (CR) formulations, (from 7 μM to 119 μM) or with an equivalent amount of respective vehicle alone. After 72 h of incubation, cells were supplemented with 10 μl of a stock solution (5 mg/ml) of MTT (Sigma) in phosphate buffer saline (PBS). At least 3 h later, culture medium was replaced with 100 μl of dimethyl-sulfoxide (DMSO, Sigma) and plates read at 560 nm with a “Microplate Reader model 450” (BioRad). Cell viability was expressed as average percentage of absorbance in treated cells versus control (culture medium alone).

### Animal Trials

One hundred and twenty male Syrian golden hamsters (Mesocricetus auratus), six weeks old and approximately 100 g weight were maintained in pathogen-free conditions with a 12 h light-dark cycle and rodent chow and tap water ad libitum. The animal-handling protocol was in accordance with the State regulations and approved by the Institutional Animal Care and Use program of the University of Turin.

According with the experimental procedure, for 14 weeks all animals right buccal pouches were painted three-weekly with 0.5% 9,10-dimethyl-1,2-benz[a]anthracene (DMBA) (Sigma) dissolved in paraffin oil, using a No. 4 natural paint-brush; the unilateral formation of tumors allowed the animals to eat and swallow normally. The amount of carcinogen and treatment delivered to each animal was quite uniform using the “wiped-brush” method [Vairaktaris et al. 2008].

Animals were randomly divided into seven groups: one “super-control” group DMBA painted alone (n=15, group D), three groups treated with RV dissolved in EtOH (n=15, group ER), complexed in HPβCD either contained in cream (n=15, group CR) or in mouthwash (n=15, group MR) respectively and three correspondent control groups vehicles without RV treated (EtOH n=10, group E; cream n=10, group C; mouthwash n=10, group M).

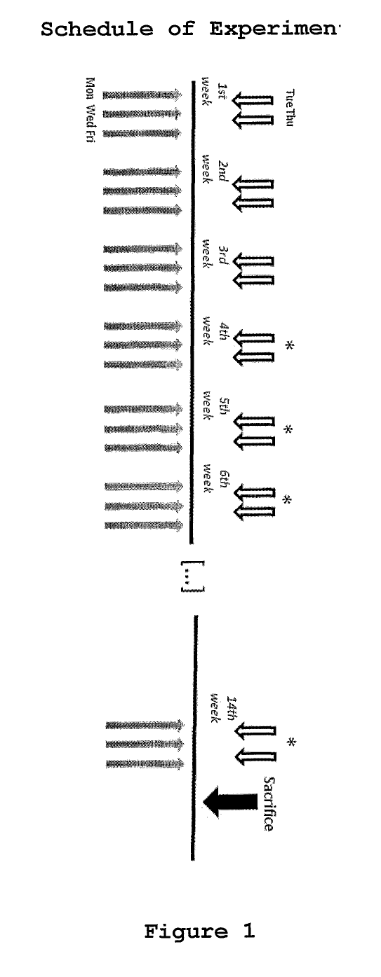

Alternating with DMBA carcinogenesis induction, twice a week (every Tuesday and Thursday) formulations with or without RV have been topically administered (FIG. 1) and performed for 5 seconds in the same DMBA painted pouches. For ER group, every application consisted in 35 μl (loadable volume on a brush No. 4) of a RV-saturated EtOH solution (65 mg/ml) topically applied on the entire pouch surface. The E group was treated in the same manner with 35 μl of EtOH alone. Consequently, the same amount of RV was carried out for both cream and mouthwash formulations contained in 140 and 250 μl respectively. Based on their viscosity, cream was administered with paint-brush at the C and CR groups, while mouthwash was applied with a syringe with a plastic spout and then pouches were shacked to mimic a rinse (M and MR groups).

Weekly, from 4^{th} week, animals were weighted and lightly anesthetized. Pouches were everted and lesions were counted, measured, scored and photographed. The end-points for data analysis included the number of animals with lesions (incidence), the number of lesions per animal (lesion multiplicity), the average lesions diameter (in millimeters) measured with a vernier caliper at two different planes, and a “pathologycal score” describing the clinical situation of the entire buccal pouch. Lesion rating was arbitrarily established considering dimension and severity of lesions. For every one, score was attributed as follows: 0 for no lesions, 1 for a leukoplakia-like lesion, 2 for a smaller than 1 mm diameter exophytic lesion (ExL), 3 to for ExL bigger than 1 mm proportionally to the increase of their mean diameter.

The last thirty animals were randomly divided in three further groups (n=10 each) to evaluate the local toxicity of the empty vehicles (TE, TC and TM) without DMBA carcinogenesis induction. Topical application of EtOH, cream and mouthwash was performed in the same manner before described.

### Histological Evaluation

Excised samples were fixed in 10% neutralized formaldehyde solution (Sigma) and embedded in paraffin. 8 μm thick sections were prepared serially from each sample. Every 80 μm, sections were mounted on xilanated glass slides and hematoxiline-eosine (HE) stained. Images were captured with an Axiovert Zeiss microscope (Zeiss, Axiovert 100 M).

### Statistical Analysis

Cytotoxicity assay data were expressed as average±standard error and experimental data were analysed with T-test (Primit Software). To test in vivo RV-Fs efficacy, we analyzed prevalence, diameter and score with T-test, whereas incidence with Fisher's exact test. The P-value is considered significant when less than 0.05.

### Results

### Vehicles Adverse Reaction and Toxicity

In vitro cytotoxicity of formulations without RV (Fs: EtOH (E), cream (C) and mouthwash (M)), was tested by MTT assay on HCPC1 cells at 24, 48 and 72 h and expressed as the average of three separate experiments. Dilution range (v/v) of Fs in cell culture complete medium, was between 1/10-1/10,000 (25-0.025 mM for HPβCD concentrations) and the data at 72 h are summarized in FIG. 2.

While EtOH has shown an anti-proliferative action at dilution <1/100 (about 170 mM) since 48 h after incubation (with similar results at 72 h, panel A, FIG. 2), C and M have demonstrated cell toxicity only after 72 h and with 1/10 dilution (panel B and C, FIG. 2); results obtained after 24 h of treatment with all vehicles and 48 h for C and M were ineffective on cell proliferation.

In vivo side effects were investigated after 14 weeks of E, C and M topical applications on right buccal pouches on three groups of animals (TE, TC and TM respectively). Gross observations of pouches and principal organs (kidney, liver, spleen and stomach) of each animal of all groups did not reveal any macroscopic alterations. Histological HE staining analysis was performed by seriate slides on the same treated pouches and organs: absence of local adverse reactions (including acute or chronic inflammations) was found.

### RV-Based Preparation Efficacy

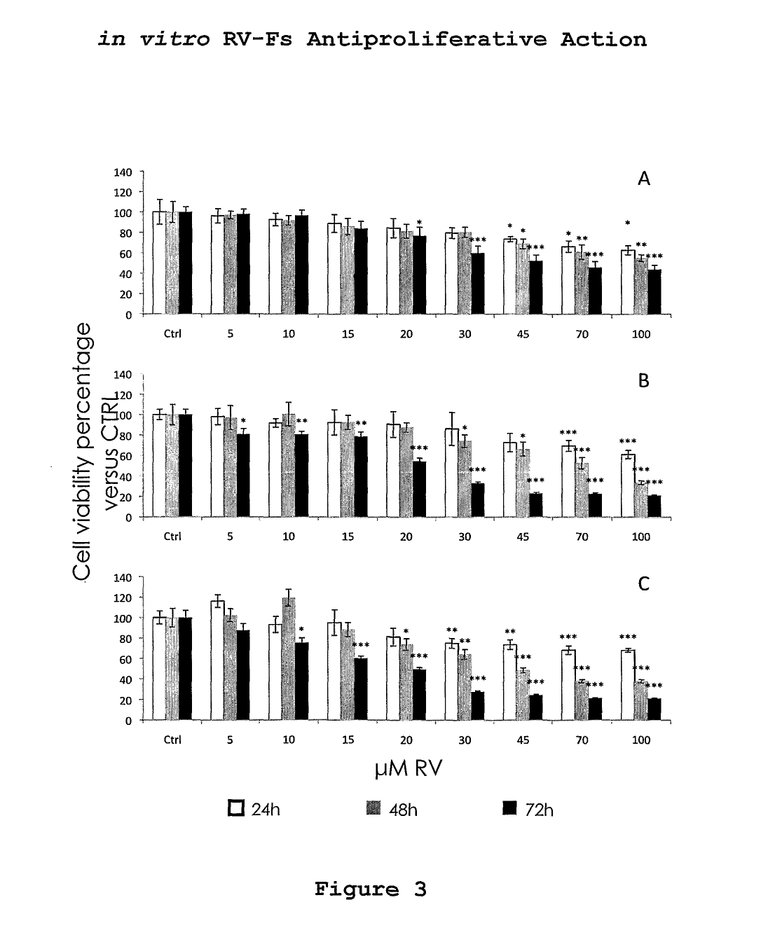

In vitro cytotoxicity of Fs containing RV (RV-Fs: EtOH (ER), cream (CR) and mouthwash (MR)) was tested by MTT assay on HCPC1 cells as described for Fs. Concentration range of RV in cell culture complete medium, was between 5-100 μM and are presented in FIG. 3. All RV-Fs cytotoxicities are concentration- and time-dependent. If not specified no significant difference was noted between groups.

ER data are shown in panel A: 50% of cell growth inhibition (EC₅₀) was achieved only after 72 h at about 45 μM concentration (p<0.001), although significant data were obtained since at 20 μM (with 25% of inhibition, p<0.05); while not reaching the 50% of growth suppression, significant 30% inhibition (p<0.05) was achieved also for 24 and 48 h (both from 45 μM). When RV was complexed with HPβCD (CR and MR formulations) requested time and concentration are earlier and lower (see FIG. 3, panel B and C) if compared with ER data. In deep, CR EC₅₀ are 70 and 20 μM at 48 and 72 h respectively (p<0.001), while for MR at corresponding time are 45 and 20 μM (p<0.001); at 24 h the EC₅₀ was not reached for both RV-Fs. Also the significative growth inhibition was generally improved in CR and MR: for the first one, it is 45, 30 and 5 μM (p<0.05) for considered times, while for the second it is 30 (p<0.01), 20 and 10 μM (p<0.05).

For tested concentrations, the maximum inhibitory effect versus control (complete medium alone) at 72 h was about 55% for ER (70-100 μM), while CR and MR displayed about 70% of inhibition since 30 μM.

In vivo experiments were designed to examine chemopreventive and tumour growth inhibition after continuous local application of RV-Fs beginning the day after the first application of the carcinogen and lasting until termination of the experiment (FIG. 1).

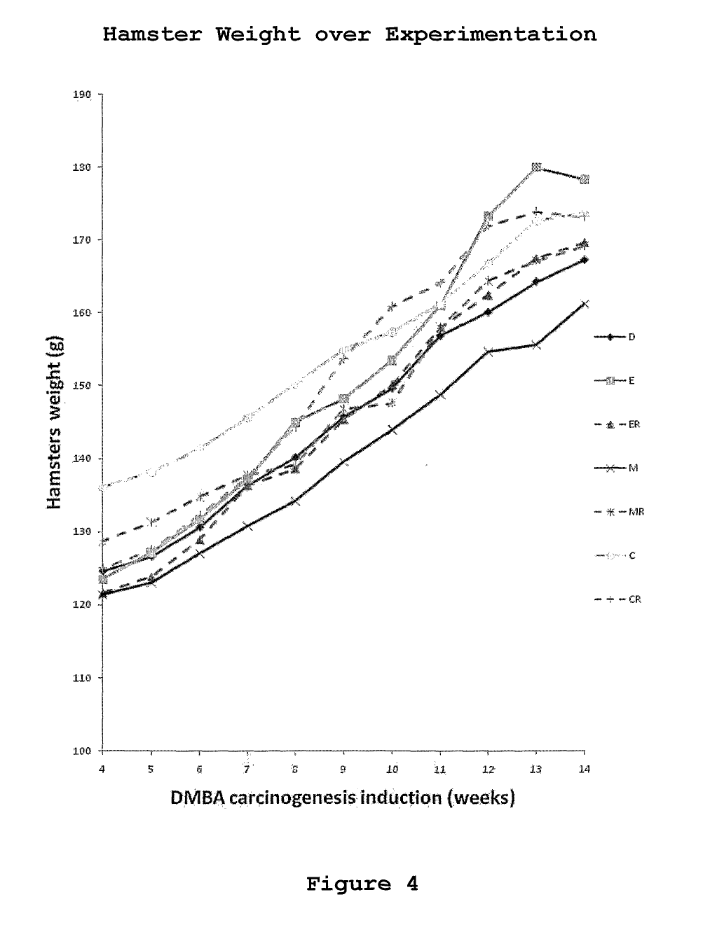

Animals appeared healthy throughout the studies, except five hamsters (respectively one belonging to D, MR, and C, while 2 to E group) which died unpredictably during the experiment and not be included. The body weight of the seven groups was monitored once a week and no significant weight differences were evidenced all along the experiment (FIG. 4). The animals were sacrificed at the 14^{th} week when all ones belonging to control groups (D, E, C, and M) displayed ExL.

General observations of pouches started at 4^{th} week: on buccal tissue of control groups DMBA induction provoked macroscopic alterations (thickened mucosa, with a rough surface and whitish granular appearance) more evident than ones occurred in RV-treated hamsters (ER, CR and MR). Already at 9^{th} week all control groups animals shown the macroscopic alteration described above, instead some of RV-treated ones have maintained the normal aspect until the experiment end.

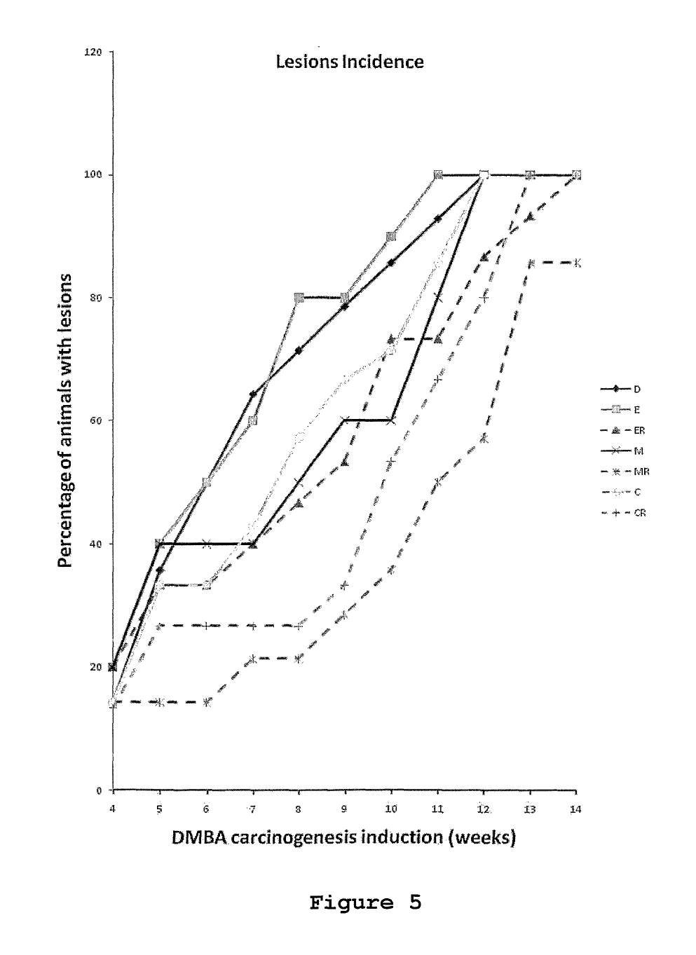

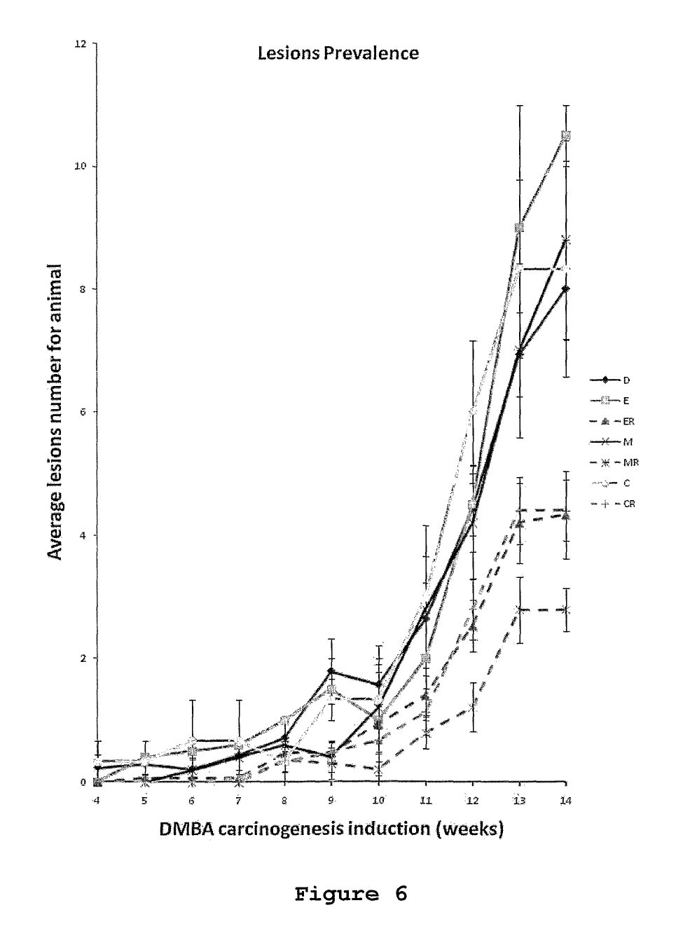

Considering animals with lesions (including OPL and ExL) RV treated groups displayed a retard in their appearance (FIG. 5): the strongest action was found in MR group since 4^{th} until 12^{th} week (p<0.05), whereas ER treatment was less effective and significance was less durable. CR group demonstrated an intermediate behaviour. The incidence was quite similar after 12^{th} week for all groups. Also the effect of RV treatment on number of lesions for animal (prevalence) has shown similar efficacy regarding MR behaviour, which had better results if compared with CR and ER groups (both p<0.05); CR and ER displayed similar pattern of profiles. Significative inhibition of lesions development was observed since 9^{th} weeks (FIG. 6) and at the end of experiment RV-treated groups had an average number three fold lesser than control groups (ER and CR p<0.01 and MR p<0.001).

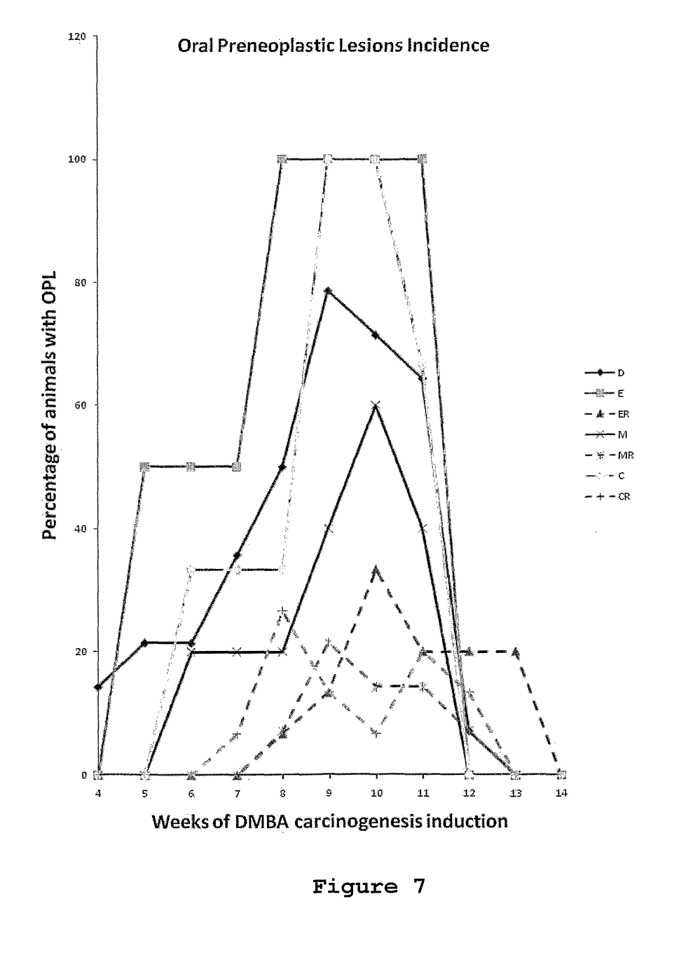

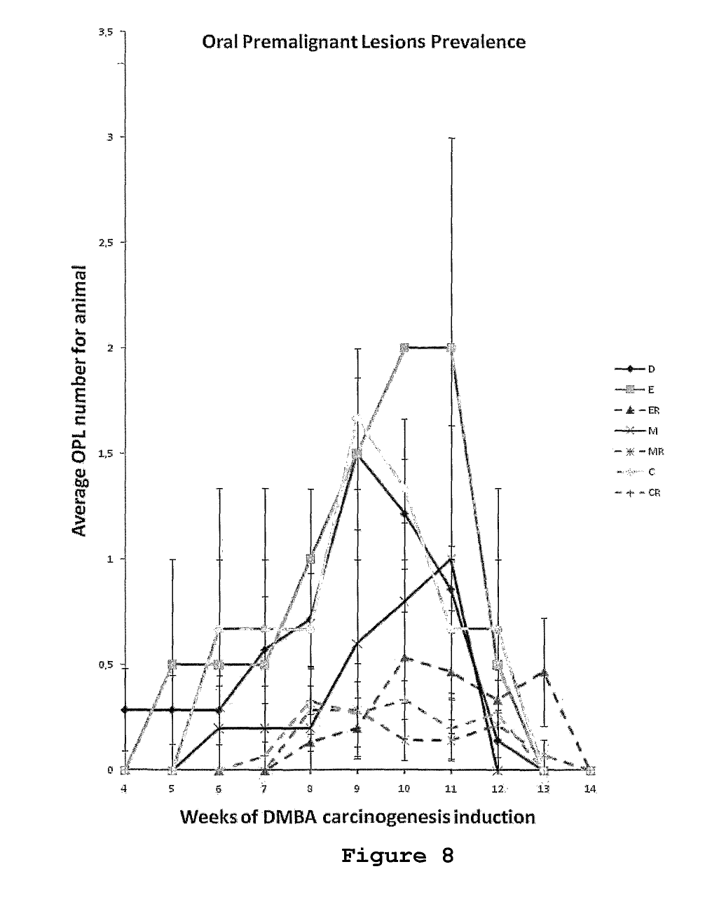

Since 4-5^{th} week of DMBA applications, the cheek pouches developed premalignant lesions with increasing incidence and prevalence until the 10-11^{th} week. After this time, both OPL data diminished for contextual presence or their evolution in ExL. However, in control groups the number of animals with OPL was higher than in RV-treated groups (60-100% vs 20-30% of incidence); time point of significance was found between 7-11^{th} week (D vs ER, CR and MR, E vs ER, C vs CR and M vs MR p<0.05; FIG. 7). In control groups the average number of lesions was at least 1, while in RV-treated was less than 0.5; in the same way, the significance persisted between 7-11^{th} week (D vs ER p<0.05, D vs CR and MR p<0.01 and D vs MR p<0.01; E vs ER p<0.01, C vs CR and M vs MR p<0.05; FIG. 8). Interestingly, OPL RV-treated groups remained for more time than controls ones.

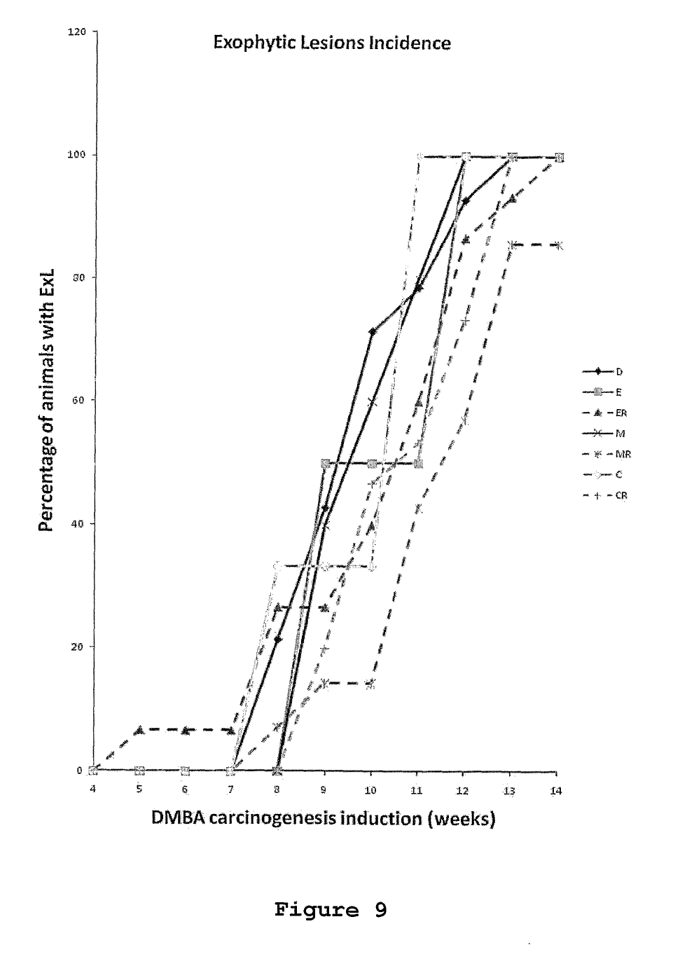

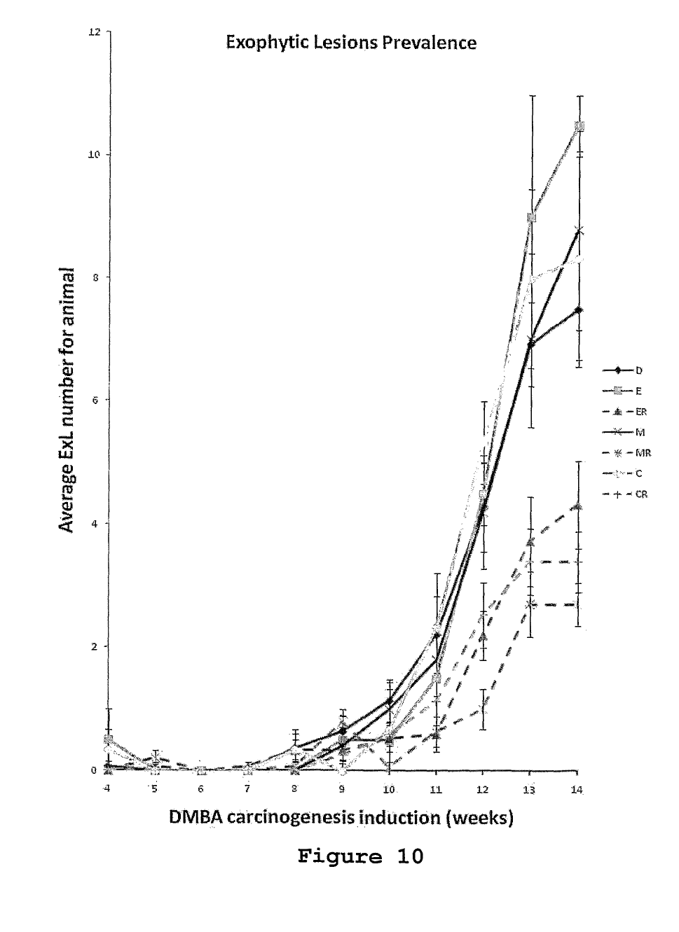

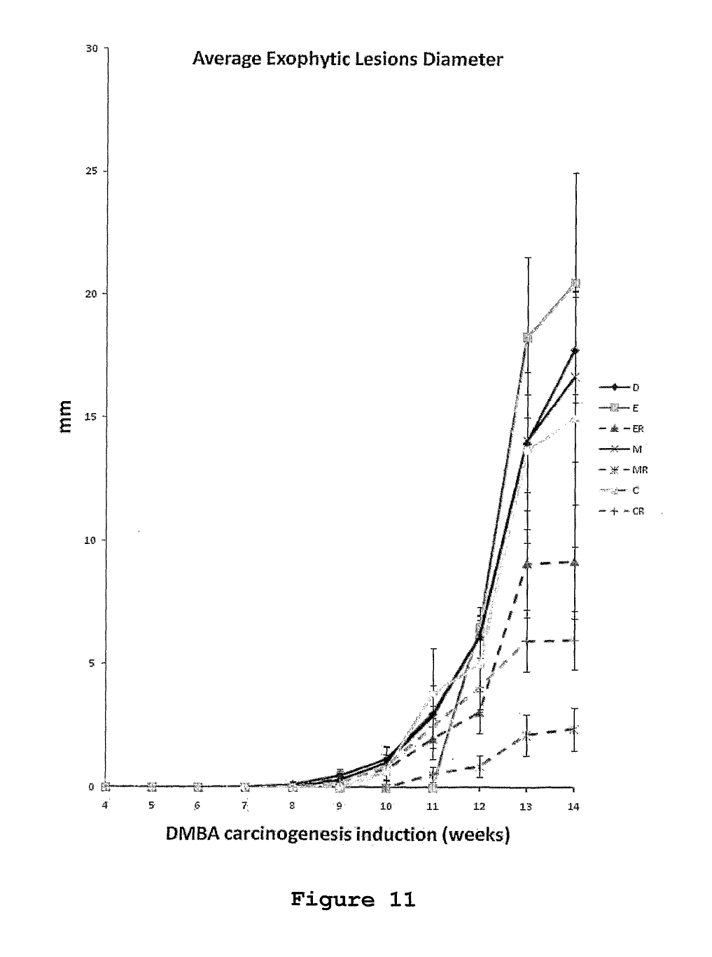

ExL incidence was not very conditioned by RV treatment (FIG. 9): few significance points were found if controls group vs RV-treated ones are compared, mainly for MR (p<0.05). ExL prevalence, instead, was very influenced by RV (FIG. 10), with similar data pattern obtained for total lesions prevalence, but with improved significance versus control groups (D vs ER and CR p<0.01, D vs MR p<0.001, E vs ER 0.05, C vs CR p<0.01 and M vs MR p<0.001); MR action is also significant if compared with ER and CR (p<0.05). Lesions average diameters (expressed in mm) was largely conditioned by RV-Fs during last three weeks: ER and CR lesions had about half the size of control ExL (p<0.05) while MR group demonstrated more efficacy with lesions 10 fold smaller (p<0.001; FIG. 11).

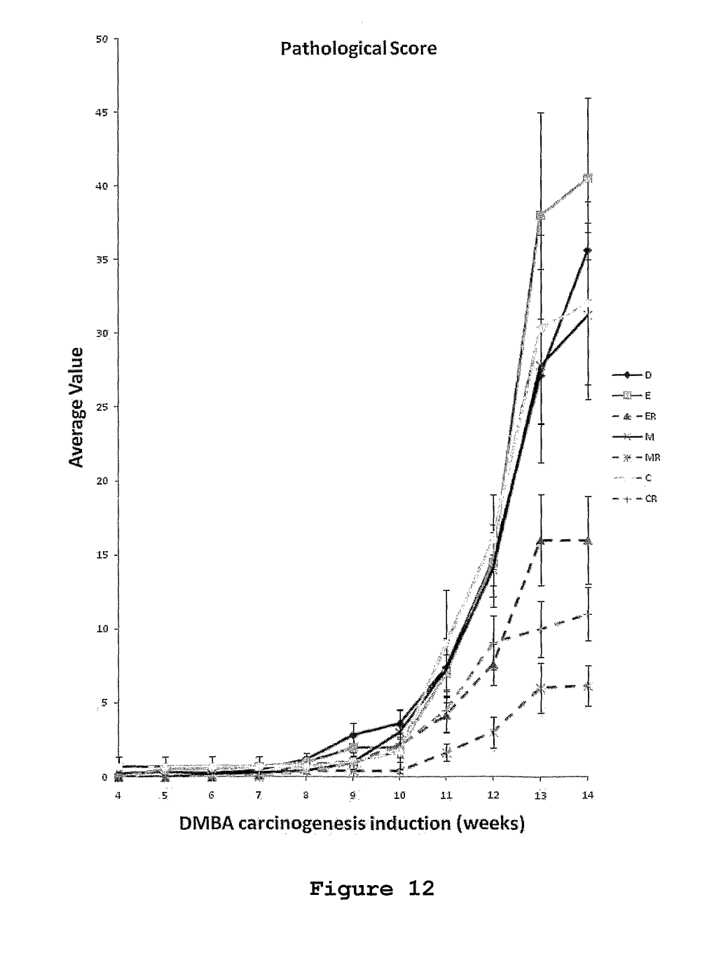

Average PS was concerned by RV-treatment especially in last weeks. Once more MR has been shown the major protective activity in lesion progression through carcinogenesis steps: since 9^{th}-10^{th} week, significant differences were evident between MR and control groups (MR vs D and M p<0.001) and, interestingly, versus the other RV-Fs (MR vs ER and CR p<0.05). Also ER and CR were significant if compared with D group and respective controls, both since 12^{th} week (for all p<0.05; FIG. 12).

Histological analysis of excised specimens (HE-stained sections) showed relevant differences between controls (D, E, C and M) and RV-Fs treated animal samples. Whereas control groups displayed tumors with analogue dimension and malignancy, RV-Fs treated ones were characterized by a less severe pathology. An overview of histological characteristics is summarized in Table 1.

TABLE 1

| | | | Horn | | Nuclear | Local |
|---|---|---|---|---|---|---|
| Group | Stroma | Vessels | pearl | Hyalinosis | atypia | malignancy |
| Controls | + | ++ | ++ | +/− | ++ | ++ |
| RV-Fs | ++/+++ | +/− | +/− | ++ | + | + |

−, absent;
+, barely present:
++, present;
+++ abundant

Within RV formulations, CR and MR treatment demonstrated a lower malignancy grade with respect to ER. Furthermore, in these groups an increasing of malignancy signs was proportional to lesion dimension. In this point of view ER group had greater and more malignant lesions with respect to CR and MR ones. Therefore, for this comparison lesions with analogue dimensions from every control and RV-Fs group were considered.

FIG. 13 (100× magnification) shows how basal membrane is generally preserved in RV-treated lesions, frequently compatible with a papilloma-like alteration. Also, in these specimens stroma deposition is present, and sometime abundant, with hyalinosis (panel A). Instead, in control lesions a reduced presence of stroma is opposed to a heavily altered structure, due to loss of basal membrane and epithelial infiltration with horn pearls development, situation often agreeable with a SCC. Moreover, lymphocytes infiltrations are present expecially in control groups, where the strong presence of inflammation leads to necrotic areas development. Nucleus analysis, however, shows alteration in morphology, such as pyknotic nucleus and bi- or tri-nucleations, abundant in control groups, but present also in smaller amounts in RV-treated ones.

Naturally, while the principle of the invention remains the same, the details of construction and the embodiments may widely vary with respect to what has been described and illustrated purely by way of example, without departing from the scope of the present invention.

### REFERENCES

Odukoya O, Schwartz J, Weichselbaum R, Shklar G. “An epidermoid carcinoma cell line derived from hamster 7,12-dimethylbenz[a]anthracene-induced buccal pouch tumors.” J Natl Cancer Inst. 1983 December; 71(6):1253-64.
Vairaktaris E, Spyridonidou S, Papakosta V, Vylliotis A, Lazaris A, Perrea D, Yapijakis C, Patsouris E. “The hamster model of sequential oral oncogenesis.” Oral Oncol. 2008 April; 44(4):315-24.

1. Pharmaceutical formulation comprising at least one aryl hydrocarbon receptor ligand in association with at least one cyclodextrin, wherein the aryl hydrocarbon receptor ligand is selected among compounds of formula I and formula II: 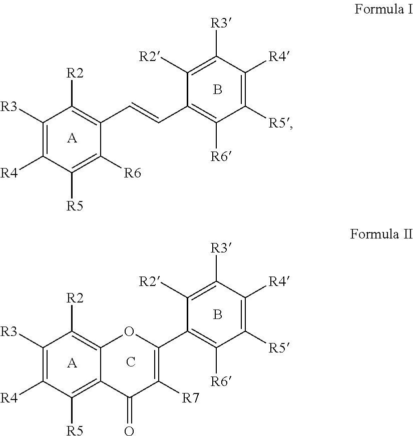
wherein
R2, R3, R4, R5, R6, R7 and R2′ R3′, R4′, R5′, R6′ are identical or different (including all symmetrical derivatives) and represent H, OH, R (where R represents substituted or unsubstituted, saturated or unsaturated, linear or branched aliphatic groups containing one to thirty carbon atoms), Ac (where Ac represents substituted or unsubstituted, saturated or unsaturated, cyclic compounds, including alicyclic and heterocyclic compounds, preferably containing three to eight atoms), Ar (where Ar represents substituted or unsubstituted, aromatic or heteroaromatic groups preferably containing five or six atoms), Cr (where Cr represents substituted or unsubstituted fused Ac and/or Ar groups, including Spiro compounds and norbornane systems, preferably containing two to five rings), OR, X (where X represents an halogen atom), CX₃, CHX₂, CH₂X, glucoside, galactoside, mannoside derivates, sulfate and glucuronide conjugates, all optical and geometrical isomeric derivatives, plant extracts containing one of these molecules, naturally or synthetically produced.

2. Pharmaceutical formulation according to claim 1, wherein the at least one aryl hydrocarbon receptor ligand is selected among resveratrol, apigenin, luteolin, tangeritin, diosmin, flavoxate, piceatannol, oxyresveratrol, quercetine, kaempferol, diosmetine, galangin, baicalein, ellipticines, chrysin and derivatives thereof.

3. Pharmaceutical formulation according to claim 1, wherein the at least aryl hydrocarbon receptor ligand is present in a concentration comprised in the range 1 to 1000 mM, preferably in the range 10 to 300 mM.

4. Pharmaceutical formulation according to claim 1, wherein the at least one cyclodextrin is selected among substituted or unsubstituted α-, β-, γ-cyclodextrins, derivatives and salts thereof.

5. Pharmaceutical formulation according to claim 4, wherein the at least one cyclodextrin is selected among hydroxyalkylated-β-cyclodextrins and hydroxyalkylated-γ-cyclodextrins, preferably 2-hydroxypropyl-β-cyclodextrin.

6. Pharmaceutical formulation according to claim 1, wherein the at least one cyclodextrin is present in an amount, expressed as percentage by weight with respect to the formulation total weight, comprised in the range 10 to 40%, preferably in the range 25 to 35%.

7. Pharmaceutical formulation according to claim 1, wherein the formulation is suitable for topical and oral administration.

8. Pharmaceutical formulation according to claim 1, wherein the formulation is suitable for skin and mucosa application.

9. Pharmaceutical formulation according to claim 1, wherein the formulation is suitable for the treatment and/or prevention of carcinomas, preferably squamous carcinomas, more preferably oral and cutaneous squamous carcinomas.

10. Pharmaceutical formulation according to claim 1, wherein the formulation is in the form of mouthwashes, creams, gels, sprays, capsules, syrups, ointments, shampoo, lotions, chewingum, tablets, candies, patches, drops, pastes, powders for recostitutions, granules, tooth-pastes.

11. Use of the pharmaceutical formulation according to claim 1 for the treatment or prevention of carcinomas, preferably squamous carcinomas, dermatitis, psoriasis, hyperkeratotic lesions, eczema, actinic dyskeratosis.
